(19) United States
(12) Patent Application Publication
(10) Pub. No.: US 2012/0090055 A1
(43) Pub. Date: Apr. 12, 2012

(54) DISPERSION OF A POLYURETHANE, CONTAINING A PESTICIDE

(75) Inventors: Karl-Heinrich Schneider, Kleinkarlbach (DE); Sven Harmsen, Tiefenthal (DE); Michael Merk, Arese (IT); Erich Birner, Altleiningen (DE); Marc Nolte, Mannheim (DE); Cedric Dieleman, Scheibenhard (FR); Alexander Kopf, Speyer (DE); Birgit Blanz, Neustadt (DE); Steffen Henkes, Böhl-Iggelheim (DE)

(73) Assignee: BASF SE, Ludwigshafen (DE)

(21) Appl. No.: 13/377,263

(22) PCT Filed: Jun. 7, 2010
(86) PCT No.: PCT/EP2010/057869

§ 371 Date: Dec. 9, 2011

(30) Foreign Application Priority Data
Jun. 12, 2009 (EP) 09162592.1

(51) Int. Cl. A01N 25/04; A01N 47/24; A01C 1/06; A01P 21/00; A01P 7/02; A01P 13/00; A01P 7/04; A01H 5/00; A01P 3/00

(52) U.S. Cl. 800/298; 504/360; 514/772.3; 514/355; 504/100

(57) ABSTRACT

The present invention relates to a method of applying an aqueous, pesticide-comprising dispersion of a polyurethane which is a reaction product of at least one polyol (A) and at least one polyisocyanate (B) to plants or plant parts. The invention furthermore relates to an aqueous pesticide-comprising dispersion of a polyurethane which is a reaction product of at least one polyol (A) and at least one polyisocyanate (B). Furthermore, it relates to the use of the dispersion for controlling phytopathogenic fungi and/or undesired plant growth and/or undesired insect or mite attack and/or for regulating the growth of plants, by allowing the dispersion to act on the respective pests, their environment and/or the plants or plant parts to be protected from the respective pests, the soil and/or on undesired plants and/or the use plants and/or their environment.

The present invention relates to a method of applying an aqueous, pesticide-comprising dispersion of a polyurethane which is a reaction product of at least one polyol (A) and at least one polyisocyanate (B) to plants or plant parts. The invention furthermore relates to an aqueous pesticide-comprising dispersion of a polyurethane which is a reaction product of at least one polyol (A) and at least one polyisocyanate (B). Furthermore, it relates to the use of the dispersion for controlling phytopathogenic fungi and/or undesired plant growth and/or undesired insect or mite attack and/or for regulating the growth of plants, by allowing the dispersion to act on the respective pests, their environment and/or the plants or plant parts to be protected from the respective pests, the soil and/or on undesired plants and/or the useful plants and/or their environment. Combinations of preferred features with other preferred features are comprised by the present invention.

Plants are not only exposed to climatic factors, but also to attack by pests. These include bacteria, yeasts, viruses, but mainly insects and harmful fungi. They exploit the surface of plants, plant parts or wounds in order to penetrate. A sufficient protection of surfaces, pores or wounds of plants is therefore necessary.

It is known that a series of plant pathogens such as bacteria, yeasts, viruses, but mainly harmful fungi, penetrate into wounds of woody parts as they are inflicted for example by the routine pruning of fruiting trees or by browsing game and by grafting methods, from where they infect all of the plant. Such an infection can lead to quality losses of the wood, to disease of the plant, or to reduced yields, indeed to the loss of the fruit-bearing capacity of the woody parts, or the dying of the plant. This damage is frequently irreversible. To avoid such infections via the wound, wounds in woody parts are, as a rule, sealed against air and water with the aid of, for example, waxy pruning compound. It has been known since the thirties of the last century to seal wounds in wood, for example pruning wounds in grapevines, with tar or a substance with disinfecting activity. A variety of resin-like substances have been employed for the surface treatment and for sealing wounds in plants. At the beginning, tar or bitumen was preferred. However, these materials become brittle over time and permit a later attack by the pathogens.

In recent times, the wood disease esca (derived from yska, which means “rotten wood” in Greek) in grapevines has increasingly led to problems in viticulture. Esca comprises a complex of fungal pathogens. The pathogens which, according to the literature, have been associated with esca symptoms are Fomitiporia punctata (syn. Phellinus punctatus), Fomitiporia mediterrana, Phaeoacremonium spp., Phaeoacremonium aleophilum and Phaemoniella chlamydosporum. A particular fungus which has been isolated from the wood of esca-infected grapevines is Fomitiporia mediterrana (heart rot fungus). The colonization of the grapevines by the pathogens takes place via injuries, in particular via pruning wounds, which are sensitive to infections over several months. The spores or conidia, which are air-borne, land on the pruning wounds and grow into the grapevines. The infestation of the wooded part spreads over several years before the first symptoms are visible. The wood rots, and the vascular bundles are destroyed. No effective protective measures against esca are known to date, with the exception of a minimization of the infection potential, by removing infected wood from the plantation. A mechanical protection of the open wounds after pruning the grapevines can be obtained by applying wound sealant to the pruning cuts, which prevents the pathogen from penetrating.

WO 07/110354 describes the use of strobilurins for the curative and for the protective treatment of esca infections.

WO 09/040339 discloses a liquid composition comprising a water-insoluble sealing agent in dissolved or dispersed form, a plant protectant, a volatile diluent and a nonionic surface-active substance in an amount of from 10 to 100% by weight, based on the sealing agent. The sealing agent may be for example a polyurethane. A disadvantage is the high surface-active substance content in the composition.

It was therefore an object of the invention to find a method of applying a protective layer to the surfaces of plants or plant parts. The method should allow simple application, for example by spraying. A further object was that the method should lead to a durable protective layer. In particular, it was an object to find a method for the protective treatment of fungal diseases on woody plants, specifically for the treatment of esca in grapevines.

The object was solved by a method of applying an aqueous, pesticide-comprising dispersion of a polyurethane to plants or plant parts, where the polyurethane is a reaction product of at least one polyol (A) and at least one polyisocyanate (B), where the polyisocyanate comprises at least 10% by weight of aromatic diisocyanate and at least 10% by weight of aliphatic diisocyanate, in each case based on the polyisocyanate.

A first subject matter of the invention therefore relates to such a method of applying an aqueous, pesticide-comprising dispersion of a polyurethane to plants or plant parts. A further subject matter is an aqueous pesticide-comprising dispersion of a polyurethane, which is a reaction product of at least one polyol (A) and at least one polyisocyanate (B), where the polyisocyanate comprises at least 10% by weight of aromatic diisocyanate and at least 10% by weight of aliphatic diisocyanate, in each case based on the polyisocyanate.

The polyurethane is usually a reaction product of at least one polyol (A) and at least one polyisocyanate (B), where the polyisocyanate comprises at least 10% by weight of aromatic diisocyanate and at least 10% by weight of aliphatic diisocyanate, in each case based on the polyisocyanate. The polyurethane is preferably a reaction product of at least one polyol, at least one polyisocyanate and at least one salt (C) of an aminocarboxylic acid or of an aminosulfonic acid, where the polyisocyanate comprises at least 10% by weight of aromatic diisocyanate and at least 10% by weight of aliphatic diisocyanate, in each case based on the polyisocyanate. The polyurethane is especially preferably a reaction product of at least one polyol, at least one polyisocyanate, at least one salt of an aminocarboxylic acid or of an aminosulfonic acid and at least one chain extender (D), which is a diol, diamine, amino alcohol or water, where the polyisocyanate comprises at least 10% by weight of aromatic diisocyanate and at least 10% by weight of aliphatic diisocyanate, in each case based on the polyisocyanate.

Suitable polyols (A) are compounds having at least 2 hydroxyl groups, such as low-molecular-weight diols or polyols and polymeric polyols such as polyester polyols, polycarbonate diols, polyacrylate polyols and polyether diols and their mixtures. With a view to good film-forming properties and elasticity, suitable polyols are predominantly higher-molecular-weight polyols with a molecular weight of approximately from 500 to 6000 g/mol, preferably of approximately from 1000 to 3000 g/mol. The polyol preferably comprises a polyesterol, in particular a polyester polyol, which is composed of aliphatic diols and aliphatic dicarboxylic acids. The polyesterol preferably has a molecular weight of below 10 000 g/mol, preferably from 500 to 6000 g/mol and in particular from 800 to 4000 g/mol.

Examples of suitable polyester polyols are the polyester polyols which are known, for example, from Ullmanns Enzyklopadie der Technischen Chemie, 4th edition, volume 19, pages 62 to 65. It is preferred to employ polyester polyols which are obtained by reacting diols with dicarboxylic acids. In the place of the dicarboxylic acids, it is also possible to use, to produce the polyester polyols, the corresponding carboxylic anhydrides or the corresponding carboxylic acid esters of lower alcohols or their mixtures. The dicarboxylic acids may be aliphatic, cycloaliphatic, araliphatic, aromatic or heterocyclic and may be optionally substituted, for example by halogen atoms, and/or unsaturated. Examples which may be mentioned are: suberic acid, azelaic acid, phthalic acid, isophthalic acid, phthalic anhydride, tetrahydrophthalic anhydride, hexahydrophthalic anhydride, tetrachlorophthalic anhydride, endomethylenetetrahydrophthalic anhydride, glutaric anhydride, maleic acid, maleic anhydride, alkenylsuccinic acid, fumaric acid, dimeric fatty acids. Preferred are aliphatic dicarboxylic acids of the general formula HOOC—(CH₂)_{y}—COOH where y is a number from 1 to 20, preferably an even number from 2 to 20, for example succinic acid, adipic acid, sebacic acid and dodecanedicarboxylic acid.

Diols which are suitable for the preparation of the polyester polyols are, for example, ethylene glycol, propane-1,2-diol, propane-1,3-diol, butane-1,3-diol, butane-1,4-diol, butene-1,4-diol, butyne-1,4-diol, pentane-1,5-diol, neopentyl glycol, bis-(hydroxymethyl)cyclohexanes such as 1,4-bis(hydroxymethyl)cyclohexane, 2-methylpropane-1,3-diol, methylpentane diols, furthermore diethylene glycol, triethylene glycol, tetraethylene glycol, polyethylene glycol, dipropylene glycol, polypropylene glycol, dibutylene glycol and polybutylene glycols. Preferred are aliphatic diols of the general formula HO—(CH₂)ₓ—OH where x is a number from 2 to 20, preferably an even number from 2 to 12. Examples are ethylene glycol, butane-1,4-diol, hexane-1,6-diol, octane-1,8-diol and dodecane-1,12-diol. Furthermore preferred are neopentyl glycol and pentane-1,5-diol.

Polyester diols which are based on lactones are also suitable, taking the form of lactone homopolymers or mixed polymers, preferably of adducts, having terminal hydroxyl groups, of lactones and suitable difunctional starter molecules. Suitable lactones are preferably those which are derived from compounds of the general formula HO—(CH₂)₂—COOH where z is a number from 1 to 20 and one H atom of a methylene unit may also be substituted by a C₁- to C₄-alkyl radical. Examples are ε-caprolactone, β-propiolactone, γ-butyrolactone and/or methyl-ε-caprolactone and their mixtures. Examples of suitable starter components are the low-molecular-weight divalent alcohols which have been mentioned above as structural component for the polyester polyols. The corresponding polymers of the ε-caprolactone are especially preferred. Others which may be employed as starters for the preparation of the lactone polymers are lower polyester diols or polyether diols. Instead of the lactone polymers, it is also possible to employ the corresponding, chemically equivalent polycondensates of the hydroxycarboxylic acids which correspond to the lactones.

Also suitable as polyols are furthermore also polycarbonate diols as can be obtained for example by reacting phosgene with an excess of the low-molecular-weight alcohols mentioned as structural components for the polyester polyols.

Others which are suitable as polyols are furthermore polyether diols. In particular, they take the form of polyether diols which can be obtained by polymerization of ethylene oxide, propylene oxide, butylene oxide, tetrahydrofuran, styrene oxide or epichlorohydrin with itself, for example in the presence or BF₃, or by an addition reaction of these compounds, optionally as a mixture or in succession, with starting components with reactive hydrogen atoms, such as alcohols or amines, for example water, ethylene glycol, propane-1,2-diol, propane-1,3-diol, 1,1-bis(4-hydroxyphenyl)propane or aniline. Especially preferred is polytetrahydrofuran, with a molar mass of 240 to 5000 g/mol, and above all 500 to 4500 g/mol. Besides, it is also possible to employ mixtures of polyester diols and polyether diols as the monomers.

Suitable polyisocyanates (B) are those of the formula X(NCO)₂ where X is an aliphatic hydrocarbon radical having 4 to 12 carbon atoms, a cycloaliphatic or aromatic hydrocarbon radical having 6 to 15 carbon atoms or an araliphatic hydrocarbon radical having 7 to 15 carbon atoms. Examples of such polyisocyanates are tetramethylene diisocyanate, hexamethylene diisocyanate (HDI), dodecamethylene diisocyanate, 1,4-diisocyanatocyclohexane, 1-isocyanato-3,5,5-trimethyl-5-isocyanatomethylcyclohexane (IPDI), 2,2-bis(4-isocyanatocyclohexyl)propane, trimethylhexane diisocyanate, 1,4-diisocyanatobenzene, 2,4-diisocyanatotoluene, 2,6-polyisoocyanatotoluene, 4,4′-diisocyanatodiphenylmethane, 2,4′-diisocyanatodiphenylmethane, p-xylylene diisocyanate, tetramethylxylylene diisocyanate (TMXDI), the isomers of bis(4-isocyanatocyclohexyl)methane (HMDI), such as the trans/trans, the cis/cis and the cis/trans isomer, and the mixtures composed of these compounds. Further examples of polyisocyanates are the biurets and cyanurates of the abovementioned diisocyanates, and oligomeric products of these diisocyanates which, in addition to the free isocyanate groups, bear further capped isocyanate groups, for example isocyanurate, biuret, urea, allophanate, uretdione or carbodiimide groups. Preferred polyisocyanates are 1-isocyanato-3,5,5-trimethyl-5-isocyanatomethylcyclohexane (IPDI), tetramethylxylylene diisocyanate (TMXDI), hexamethylene diisocyanate (HDI) and bis-(4-isocyanatocyclohexyl)methane (HMDI). Mixtures of these isocyanates are also suitable, for example, mixtures of the respective structural isomers of diisocyanatotoluene and diisocyanatodiphenylmethane, for example a mixture of 80 mol % of 2,4-diisocyanatotoluene and 20 mol % of 2,6-diisocyanatotoluene, mixtures of aromatic isocyanates such as 2,4-diisocyanatotoluene and/or 2,6-diisocyanatotoluene with aliphatic or cycloaliphatic isocyanates such as hexamethylene diisocyanate or IPDI. The molar ratio of aliphatic or cycloaliphatic isocyanates to the aromatic isocyanates is, in most cases, 10:1 to 1:10, preferably 1:2 to 1:6.

Suitable salts (C) of an aminocarboxylic acid or an aminosulfonic acid are salts of aliphatic aminocarboxylic acids or salts of aliphatic aminosulfonic acids. Preferred are the alkali metal salts, in particular the sodium and potassium salts, of the adducts of lower aliphatic primary diamines, for example ethylene diamine, and unsaturated carboxylic acids such as (meth)acrylic acid, crotonic acid or maleic acid, and alkali metal salts of lysine. The alkali metal salts of the adducts of propanesulfone and aliphatic primary diamines are also well suited. Especially preferred are the salts of the aliphatic aminocarboxylic acids, in particular the adducts of ethylene diamine and unsaturated, aliphatic carboxylic acid salts, such as (meth)acrylates.

The salts of an aminocarboxylic acid or an aminosulfonic acid would, in most cases, be present in amounts of from 0.01 to 2% by weight, preferably from 0.05 to 1% by weight, based on the polyurethane. Polyurethanes which comprise salts (C) are generally known and described, for example, in GB1584865, GB1339357 or GB1329565.

A suitable chain extender (D) is a diol, diamine, amino alcohol or water, preferably a diol. Diols are, for example, glycols such as ethylene glycol, propylene glycol, butane-1,3-diol, butane-1,4-diol, hexane-1,6-diol, neopentyl glycol, cyclohexanediol, 2,2-bis(4-hydroxycyclohexyl)propane, 2,2-bis(4-hydroxyethoxyphenyl)propane, diethylene glycol or dipropylene glycol, preferably butane-1,4-diol or neopentyl glycol. Diamines are, for example, ethylenediamine, hydrazine, piperazine, isophorone diamine, toluenediamine or diaminodiphenylmethane.

The isocyanate groups and the hydroxyl and amino groups capable of reacting with isocyanate should be employed in approximately equivalent molar ratios. The ratio of the number of the isocyanate groups to the number of the total of hydrogen atoms capable of reacting with isocyanate should be in the range of between 0.9 and 1.2, preferably between 1.0 and 1.1. Components A, B, C and D should be employed in such molar ratios that the ratio of (A) to (B) and the total of (C) and (D) is in the range of A:B:(C+D)=1:2:1 to 1:14:13. The range of from 1:4:3 to 1:10:9 is especially advantageous.

The polyurethane is prepared in a manner known per se (for example as described in GB1584865, GB1339357 or GB1329565) by reacting the polyols (A) with the polyisocyanates (B) in the melt or in the presence of a water-miscible inert organic solvent with a boiling point below 100° C. (such as acetone, tetrahydrofuran or methyl ethyl ketone), optionally under pressure, to give a prepolymer with terminal isocyanate groups. Here, the polyisocyanates can be reacted in succession with (A) and the chain extender (D) either as a mixture with each other or else in the abovementioned sequence. When employing mixtures of (cyclo)aliphatic and aromatic polyisocyanates, it will frequently suffice to employ (B) as a mixture with each other. If they are reacted in succession with A and D, it is advantageous first to employ the aromatic and then the (cyclo)aliphatic isocyanate in order to ensure that the reaction product features central segments of aromatic diisocyanate and a chain extender as well as terminal (cyclo)aliphatic isocyanate groups. In the case of a stepwise reaction of the mixtures of (cyclo)aliphatic and aromatic polyisocyanates, it is not essential that the aromatic diisocyanate be fully reacted before adding the (cyclo)aliphatic diisocyanate, but the (cyclo)aliphatic diisocyanate may frequently already be added at the point in time at which only part of the aromatic diisocyanate has reacted. The resulting polyurethane with terminal aliphatic or cycloaliphatic isocyanate groups is optionally diluted (further) with a water-miscible solvent with a boiling point of below 100° C. which is inert towards isocyanate groups and treated with a solution, preferably an aqueous solution, of the salts (C) at a temperature of between 20 and 50° C. The reaction of the salts (C) with the isocyanate groups takes place spontaneously and leads to the chain being extended. Water may be stirred into the solution of the resulting polyurethane with incorporated salt-like groups, and the organic solvent can be removed by distillation. This gives finely divided, stable dispersions which can be concentrated by evaporation.

In general, solvent-free dispersions with a solids content of from 20 to 60% by weight, especially 30-50% by weight, are preferred. Known catalysts such as dibutyltin dilaurate, tin(II) octoate or 1,II-diazabicyclo-(2,2,2)-octane, may be used to accelerate the reaction of the polyisocyanates.

The dispersion of a polyurethane may be present as an emulsion or suspension; preferably, the polyurethane is suspended. As a rule, the polyurethane particles have a particle size distribution with a D50 value of from 0.05 to 10 μm, preferably from 0.1 to 5 μm, it being possible for the D50 value to be determined by dynamic light scattering.

The aqueous, pesticide-comprising dispersion may comprise any pesticides. The term pesticide refers to at least one active substance selected from the group of the fungicides, insecticides, nematicides, herbicides, rodenticides, safeners and/or growth regulators. Preferred pesticides are fungicides, insecticides, rodenticides and herbicides. Mixtures of pesticides from two or more of the abovementioned classes may also be used. A person skilled in the art is familiar with such pesticides, which can be found, for example, in Pesticide Manual, 14th Ed. (2006), The British Crop Protection Council, London.

Suitable fungicides are:

### A) Strobilurins:

azoxystrobin, dimoxystrobin, enestroburin, fluoxastrobin, kresoxim-methyl, metominostrobin, orysastrobin, picoxystrobin, pyraclostrobin, pyribencarb, trifloxystrobin, N-methyl-2-(2-(6-(3-chloro-2-methylphenoxy)-5-fluoropyrimidin-4-yloxy)phenyl)-2-methoxyiminoacetamide, 2-(ortho-(2,5-dimethylphenyloxymethylene)phenyl)-3-methoxyacrylic acid methyl ester, 3-methoxy-2-(2-(N-(4-methoxyphenyl)cyclopropanecarboximidoylsulfanylmethyl)phenyl)acrylic acid methyl ester, N-methyl-2-(2-(3-(2,6-dichlorophenyl)-1-methylallylideneaminooxymethyl)phenyl)-2-methoxyiminoacetamide;

### B) Carboxamides:

carboanilides: benalaxyl, benalaxyl-M, benodanil, bixafen, boscalid, carboxin, fenfuram, fenhexamid, flutolanil, furametpyr, isopyrazam, isotianil, kiralaxyl, mepronil, metalaxyl, metalaxyl-M (mefenoxam), ofurace, oxadixyl, oxycarboxin, penthiopyrad, tecloftalam, thifluzamide, tiadinil, 2-amino-4-methylthiazole-5-carboxanilide, 2-chloro-N-(1,1,3-trimethylindan-4-yl)nicotinamide, N-(2′,4′-difluorobiphenyl-2-yl)-3-difluoromethyl-1-methyl-1H-pyrazole-4-carboxamide, N-(2′,4′-dichlorobiphenyl-2-yl)-3-difluoromethyl-1-methyl-1H-pyrazole-4-carboxamide, N-(2′,5′-difluorobiphenyl-2-yl)-3-difluoromethyl-1-methyl-1H-pyrazole-4-carboxamide, N-(2′,5′-dichlorobiphenyl-2-yl)-3-difluoromethyl-1-methyl-1H-pyrazole-4-carboxamide, N-(3′,5′-difluorobiphenyl-2-yl)-3-difluoromethyl-1-methyl-1H-pyrazole-4-carboxamide, N-(3′,5′-dichlorobiphenyl-2-yl)-3-difluoromethyl-1-methyl-1H-pyrazole-4-carboxamide, N-(3′-fluorobiphenyl-2-yl)-3-difluoromethyl-1-methyl-1H-pyrazole-4-carboxamide, N-(3′-chlorobiphenyl-2-yl)-3-difluoromethyl-1-methyl-1H-pyrazole-4-carboxamide, N-(2′-fluorobiphenyl-2-yl)-3-difluoromethyl-1-methyl-1H-pyrazole-4-carboxamide, N-(2′chlorobiphenyl-2-yl)-3-difluoromethyl-1-methyl-1H-pyrazole-4-carboxamide, N-(3′,4′,5′-trifluorobiphenyl-2-yl)-3-difluoromethyl-1-methyl-1H-pyrazole-4-carboxamide, N-(2′,4′,5′-trifluorobiphenyl-2-yl)-3-difluoromethyl-1-methyl-1H-pyrazole-4-carboxamide, N-[2-(1,1,2,3,3,3-hexafluoropropoxy)phenyl]-3-diflouromethyl-1-methyl-1H-pyrazole-4-carboxamide, N-[2-(1,1,2,2-tetrafluoroethoxy)phenyl]-3-difluoromethyl-1-methyl-1H-pyrazole-4-carboxamide, N-(4′-trifluoromethylthiobiphenyl-2-yl)-3-difluoromethyl-1-methyl-1H-pyrazole-4-carboxamide, N-(2-(1,3-dimethylbutyl)phenyl)-1,3-dimethyl-5-fluoro-1H-pyrazole-4-carboxamide, N-(2-(1,3,3-trimethylbutyl)phenyl)-1,3-dimethyl-5-fluoro-1H-pyrazole-4-carboxamide, N-(4′-chloro-3′,5′-difluorobiphenyl-2-yl)-3-difluoromethyl-1-methyl-1H-pyrazole-4-carboxamide, N-(4′-chloro-3′,5′-difluorobiphenyl-2-yl)-3-trifluoromethyl-1-methyl-1H-pyrazole-4-carboxamide, N-(3′,4′-dichloro-5′-fluorobiphenyl-2-yl)-3-trifluoromethyl-1-methyl-1H-pyrazole-4-carboxamide, N-(3′,5′-difluoro-4′-methylbiphenyl-2-yl)-3-difluoromethyl-1-methyl-1H-pyrazole-4-carboxamide, N-(3′,5′-difluoro-4′-methylbiphenyl-2-yl)-3-trifluoromethyl-1-methyl-1H-pyrazole-4-carboxamide, N-(2-bicyclopropyl-2-ylphenyl)-3-difluoromethyl-1-methyl-1H-pyrazole-4-carboxamide, N-(cis-2-bicyclopropyl-2-ylphenyl)-3-difluoromethyl-1-methyl-1H-pyrazole-4-carboxamide, N-(trans-2-bicyclopropyl-2-ylphenyl)-3-difluoromethyl-1-methyl-1H-pyrazole-4-carboxamide, N-[1,2,3,4-tetrahydro-9-(1-methylethyl)-1,4-methanonaphthalen-5-yl]-3-(difluoromethyl)-1-methyl-1H-pyrazole-4-carboxamide;
carboxylic acid morpholides: dimethomorph, flumorph;
benzamides: flumetover, fluopicolide, fluopyram, zoxamide, N-(3-ethyl-3,5,5-trimethylcyclohexyl)-3-formylamino-2-hydroxybenzamide;
other carboxamides: carpropamid, diclocymet, mandipropamid, oxytetracyclin, silthiofam, N-(6-methoxypyridin-3-yl)cyclopropanecarboxamide;

### C) Azoles

triazoles: azaconazole, bitertanol, bromuconazole, cyproconazole, difenoconazole, diniconazole, diniconazole-M, epoxiconazole, fenbuconazole, fluquinconazole, flusilazole, flutriafol, hexaconazole, imibenconazole, ipconazole, metconazole, myclobutanil, oxpoconazole, paclobutrazol, penconazole, propiconazole, prothioconazole, simeconazole, tebuconazole, tetraconazole, triadimefon, triadimenol, triticonazole, uniconazole, 1-(4-chlorophenyl)-2-([1,2,4]triazol-1-yl)cycloheptanol;
imidazoles: cyazofamid, imazalil, imazalil sulfate, pefurazoate, prochloraz, triflumizole;
benzimidazoles: benomyl, carbendazim, fuberidazole, thiabendazole;
others: ethaboxam, etridiazole, hymexazole, 2-(4-chlorophenyl)-N-[4-(3,4-dimethoxypheny)isoxasol-5-yl]-2-prop-2-ynyloxyacetamide;
   D) Nitrogen-comprising heterocyclyl compounds
pyridines: fluazinam, pyrifenox, 3-[5-(4-chlorophenyl)-2,3-dimethylisoxazolidin-3-yl]pyridine, 3-[5-(4-methylphenyl)-2,3-dimethylisoxazolidin-3-yl]pyridine, 2,3,5,6-tetrachloro-4-methanesulfonylpyridine, 3,4,5-trichloropyridine-2,6-dicarbonitrile, N-(1-(5-bromo-3-chloropyridin-2-yl)ethyl)-2,4-dichloronicotinamide, N-((5-bromo-3-chloropyridin-2-yl)methyl)-2,4-dichloronicotinamide;
pyrimidines: bupirimate, cyprodinil, diflumetorim, fenarimol, ferimzone, mepanipyrim, nitrapyrin, nuarimol, pyrimethanil;
piperazines: triforine;
pyrroles: fludioxonil, fenpiclonil;
morpholines: aldimorph, dodemorph, dodemorph acetate, fenpropimorph, tridemorph;
piperidines: fenpropidin;
dicarboximides: fluorimide, iprodione, procymidone, vinclozolin;
nonaromatic 5-heterocyclic rings: famoxadone, fenamidon, octhilinone, probenazole, 5-amino-2-isopropyl-3-oxo-4-ortho-tolyl-2,3-dihydropyrazole-1-thiocarboxylic acid S-allyl ester;
others: acibenzolar-S-methyl, amisulbrom, anilazin, blasticidin-S, captafol, captan, quinomethionate, dazomet, debacarb, diclomezine, difenzoquat, difenzo-quat-methylsulfate, fenoxanil, folpet, oxolinic acid, piperalin, proquinazid, pyroquilon, quinoxyfen, triazoxide, tricyclazole, 2-butoxy-6-iodo-3-propylchromen-4-one, 5-chloro-1-(4,6-dimethoxypyrimidin-2-yl)-2-methyl-1H-benzoimidazole, 5-chloro-7-(4-methyl-piperidin-1-yl)-6-(2,4,6-trifluorophenyl)-[1,2,4]triazolo[1,5-a]pyrimidine, 6-(3,4-dichlorophenyl)-5-methyl-[1,2,4]tri-azolo[1,5-a]pyrimidin-7-ylamine, 6-(4-tert-butylphenyl)-5-methyl-[1,2,4]tri-azolo[1,5-a]pyrimidin-7-ylamine, 5-methyl-6-(3,5,5-trimethyl-hexyl)-[1,2,4]triazolo[1,5-a]pyrimidin-7-ylamine, 5-methyl-6-octyl-[1,2,4]triazolo[1,5-a]pyrimidin-7-ylamine, 6-methyl-5-octyl-[1,2,4]triazolo[1,5-a]pyrimidin-7-yl-amine, 6-ethyl-5-octyl-[1,2,4]triazolo[1,5-a]pyrimidin-7-ylamine, 5-ethyl-6-octyl-[1,2,4]triazolo[1,5-a]pyrimidin-7-ylamine, 5-ethyl-6-(3,5,5-trimethylhexyl)-[1,2,4]triazolo[1,5-a]pyrimidin-7-ylamine, 6-octyl-5-propyl-[1,2,4]triazolo-[1,5-a]pyrimidin-7-ylamine, 5-methoxymethyl-6-octyl-[1,2,4]triazolo[1,5-a]pyrimidin-7-ylamine, 6-octyl-5-trifluoromethyl-[1,2,4]triazolo[1,5-a]pyrimidin-7-ylamine and 5-trifluoromethyl-6-(3,5,5-trimethylhexyl)-[1,2,4]triazolo[1,5-a]pyrimidin-7-ylamine;
   E) Carbamates and dithiocarbamates
thio- and dithiocarbamates: ferbam, mancozeb, maneb, metam, methasulfocarb, metiram, propineb, thiram, zineb, ziram;
carbamates: diethofencarb, benthiavalicarb, iprovalicarb, propamocarb, propamocarb hydrochloride, valiphenal, 4-fluorophenyl N-(1-(1-(4-cyanophenyl)ethanesulfonyl)but-2-yl)carbamate;
   F) Other fungicides
guanidines: dodine, dodine (free base), guazatine, guazatine acetate, iminoctadine, iminoctadine triacetate, iminoctadine tris(albesilate);
antibiotics: kasugamycin, kasugamycin hydrochloride hydrate, polyoxins, streptomycin, validamycin A;
nitrophenyl derivatives: binapacryl, dicloran, dinobuton, dinocap, nitrothal-isopropyl, tecnazene;
organometallic compounds: fentin salts, such as, for example, fentin acetate, fentin chloride, fentin hydroxide;
sulfur-comprising heterocyclyl compounds: dithianon, isoprothiolane;
organophosphorus compounds: edifenphos, fosetyl, fosetyl-aluminum, iprobenfos, phosphorous acid and its salts, pyrazophos, tolclofos-methyl;
organochlorine compounds: chlorthalonil, dichlofluanid, dichlorphen, flusulfamid, hexachlorbenzene, pencycuron, pentachlorophenol and its salts, phthalide, quintozene, thiophanate-methyl, tolylfluanid, N-(4-chloro-2-nitrophenyl)-N-ethyl-4-methylbenzenesulfonamide;
inorganic active ingredients: phosphorous acid and its salts, Bordeaux mixture, copper salts, such as, for example, copper acetate, copper hydroxide, copper oxychloride, basic copper sulfate, sulfur;
others: biphenyl, bronopol, cyflufenamid, cymoxanil, diphenylamine, metrafenone, mildiomycin, oxine-copper, prohexadione-calcium, spiroxamine, tolylfluanid, N-(cyclopropylmethoxyimino-(6-difluoromethoxy-2,3-difluorophenyOmethyl)-2-phenylacetamide, N′-(4-(4-chloro-3-trifluoromethylphenoxy)-2,5-dimethylphenyl)-N-ethyl-N-methylformamidine, N′-(4-(4-fluoro-3-trifluoromethylphenoxy)-2,5-dimethylphenyl)-N-ethyl-N-methylformamidine, N′-(2-methyl-5-trifluoromethyl-4-(3-trimethylsilanylpropoxy)phenyl)-N-ethyl-N-methylformamidine, N′-(5-difluoromethyl-2-methyl-4-(3-trimethylsilanylpropoxy)phenyl)-N-ethyl-N-methyl formamidine.

Suitable growth regulators are:

abscisic acid, amidochlor, ancymidole, 6-benzylaminopurine, brassinolide, butralin, chlormequat (chlormequat chloride), choline chloride, cyclanilide, daminozide, dikegulac, dimethipin, 2,6-dimethylpuridine, ethephon, flumetralin, flurprimidole, fluthiacet, forchlorfenuron, gibberellic acid, inabenfid, indole-3-acetic acid, maleic hydrazide, mefluidid, mepiquat (mepiquat chloride), metconazole, naphthalene acetic acid, N-6-benzyladenine, paclobutrazole, prohexadione (prohexadione-calcium), prohydrojasmone, thidiazuron, triapenthenol, tributyl phosphorotrithioate, 2,3,5-tri-iodobenzoic acid, trinexapac-ethyl and uniconazole.
Suitable herbicides are:
acetamides: acetochlor, alachlor, butachlor, dimethachlor, dimethenamid, flufenacet, mefenacet, metolachlor, metazachlor, napropamide, naproanilide, pethoxamid, pretilachlor, propachlor, thenylchlor;
amino acid analogs: bilanafos, glyphosate, glufosinate, sulfosate; aryloxyphenoxypropionates: clodinafop, cyhalofop-butyl, fenoxaprop, fluazifop, haloxyfop, metamifop, propaquizafop, quizalofop, quizalofop-P-tefuryl;
bipyridyls: diquat, paraquat;
carbamates and thiocarbamates: asulam, butylate, carbetamide, desmedipham, dimepiperate, eptam (EPTC), esprocarb, molinate, orbencarb, phenmedipham, prosulfocarb, pyributicarb, thiobencarb, tri-allate;
cyclohexanediones: butroxydim, clethodim, cycloxydim, profoxydim, sethoxydim, tepraloxydim, tralkoxydim;
dinitroanilines: benfluralin, ethalfluralin, oryzalin, pendimethalin, prodiamine, trifluralin;
diphenyl ethers: acifluorfen, aclonifen, bifenox, diclofop, ethoxyfen, fomesafen, lactofen, oxyfluorfen;
hydroxybenzonitriles: bromoxynil, dichlobenil, ioxynil;
imidazolinones: imazamethabenz, imazamox, imazapic, imazapyr, imazaquin, imazethapyr;
phenoxyacetic acids: clomeprop, 2,4-dichlorophenoxyacetic acid (2,4-D), 2,4-DB, dichlorprop, MCPA, MCPA-thioethyl, MCPB, mecoprop;
pyrazines: chloridazon, flufenpyr-ethyl, fluthiacet, norflurazon, pyridate;
pyridines: aminopyralid, clopyralid, diflufenican, dithiopyr, fluridone, fluroxypyr, picloram, picolinafen, thiazopyr;
sulfonyl ureas: amidosulfuron, azimsulfuron, bensulfuron, chlorimuron-ethyl, chlorsulfuron, cinosulfuron, cyclosulfamuron, ethoxysulfuron, flazasulfuron, flucetosulfuron, flupyrsulfuron, foramsulfuron, halosulfuron, imazosulfuron, iodosulfuron, mesosulfuron, metsulfuron-methyl, nicosulfuron, oxasulfuron, primisulfuron, prosulfuron, pyrazosulfuron, rimsulfuron, sulfometuron, sulfosulfuron, thifensulfuron, triasulfuron, tribenuron, trifloxysulfuron, triflusulfuron, tritosulfuron, 1-((2-chloro-6-propyl-imidazo[1,2-b]pyridazin-3-yOsulfonyl)-3-(4,6-dimethoxy-pyrimidin-2-yl)urea;
triazines: ametryn, atrazine, cyanazine, dimethametryn, ethiozine, hexazinone, metamitron, metribuzine, prometryn, simazine, terbuthylazine, terbutryn, triaziflam;
ureas: chlorotoluron, daimuron, diuron, fluometuron, isoproturon, linuron, methabenzthiazuron, tebuthiuron;
other acetolactate synthase inhibitors: bispyribac-sodium, cloransulam-methyl, diclosulam, florasulam, flucarbazone, flumetsulam, metosulam, ortho-sulfamuron, penoxsulam, propoxycarbazone, pyribambenz-propyl, pyribenzoxim, pyriftalid, pyriminobac-methyl, pyrimisulfan, pyrithiobac, pyroxasulfon, pyroxsulam;
others: amicarbazone, aminotriazole, anilofos, beflubutamid, benazolin, bencarbazone, benfluresate, benzofenap, bentazone, benzobicyclon, bromacil, bromobutide, butafenacil, butamifos, cafenstrole, carfentrazone, cinidon-ethlyl, chlorthal, cinmethylin, clomazone, cumyluron, cyprosulfamide, dicamba, difenzoquat, diflufenzopyr, Drechslera monoceras, endothal, ethofumesate, etobenzanide, fentrazamide, flumiclorac-pentyl, flumioxazin, flupoxam, fluorochloridone, flurtamon, indanofan, isoxaben, isoxaflutole, lenacil, propanil, propyzamide, quinclorac, quinmerac, mesotrione, methylarsenic acid, naptalam, oxadiargyl, oxadiazone, oxaziclomefon, pentoxazon, pinoxaden, pyraclonil, pyraflufen-ethyl, pyrasulfotole, pyrazoxyfen, pyrazolynate, quinoclamine, saflufenacil, sulcotrione, sulfentrazone, terbacil, tefuryltrione, tembotrione, thiencarbazone, topramezone, 4-hydroxy-3-[2-(2-methoxyethoxymethyl)-6-trifluoromethylpyridine-3-carbonyl]-bicyclo[3.2.1]oct-3-en-2-one, ethyl (3-[2-chloro-4-fluoro-5-(3-methyl-2,6-dioxo-4-trifluoromethyl-3,6-dihydro-2H-pyrimidin-1-yl)-phenoxy]-pyridin-2-yloxy)acetate, methyl 6-amino-5-chloro-2-cyclopropylpyrimidine-4-carboxylate, 6-chloro-3-(2-cyclopropyl-6-methylphenoxy)-pyridazin-4-ol, 4-amino-3-chloro-6-(4-chlorophenyl)-5-fluoropyridine-2-carboxylic acid, methyl 4-amino-3-chloro-6-(4-chloro-2-fluoro-3-methoxyphenyl)pyridine-2-carboxylate and methyl 4-amino-3-chloro-6-(4-chloro-3-dimethylamino-2-fluorophenyl)pyridine-2-carboxylate.
   Suitable insecticides are:
organo(thio)phosphates: acephate, azamethiphos, azinphos-methyl, chlorpyrifos, chlorpyrifos-methyl, chlorfenvinphos, diazinon, dichlorvos, dicrotophos, dimethoate, disulfoton, ethion, fenitrothion, fenthion, isoxathion, malathion, methamidophos, methidathion, methyl-parathion, mevinphos, monocrotophos, oxydemeton-methyl, paraoxon, parathion, phenthoate, phosalone, phosmet, phosphamidon, phorate, phoxim, pirimiphos-methyl, profenofos, prothiofos, sulprophos, tetrachlorvinphos, terbufos, triazophos, trichlorfon;
carbamates: alanycarb, aldicarb, bendiocarb, benfuracarb, carbaryl, carbofuran, carbosulfan, fenoxycarb, furathiocarb, methiocarb, methomyl, oxamyl, pirimicarb, propoxur, thiodicarb, triazamate;
pyrethroids: allethrin, bifenthrin, cyfluthrin, cyhalothrin, cyphenothrin, cyper-methrin, alpha-cypermethrin, beta-cypermethrin, zeta-cypermethrin, deltamethrin, esfenvalerate, etofenprox, fenpropathrin, fenvalerate, imiprothrin, lambda-cyhalothrin, permethrin, prallethrin, pyrethrin I and II, resmethrin, silafluofen, tau-fluvalinate, tefluthrin, tetramethrin, tralomethrin, transfluthrin, profluthrin, dimefluthrin,
insect growth inhibitors: a) chitin synthesis inhibitors: benzoylureas: chlorfluazuron, cyramazin, diflubenzuron, flucycloxuron, flufenoxuron, hexaflumuron, lufenuron, novaluron, teflubenzuron, triflumuron; buprofezin, diofenolan, hexythiazox, etoxazole, clofentazin; b) ecdysone antagonists: halofenozide, methoxyfenozide, tebufenozide, azadirachtin; c) juvenoids: pyriproxyfen, methoprene, fenoxycarb; d) lipid biosynthesis inhibitors: spirodiclofen, spiromesifen, spirotetramate;
nicotin receptor agonists/antagonists: clothianidin, dinotefuran, imidacloprid, thiamethoxam, nitenpyram, acetamiprid, thiacloprid, 1-(2-chlorothiazol-5-ylmethyl)-2-nitrimino-3,5-dimethyl-[1,3,5]triazinane;
GABA antagonists: endosulfan, ethiprole, fipronil, vaniliprole, pyrafluprole, pyriprole, 5-amino-1-(2,6-dichloro-4-methylphenyl)-4-sulfinamoyl-1H-pyrazole-3-thiocarboxamide;
macrocyclic lactones: abamectin, emamectin, milbemectin, lepimectin, spinosad, spinetoram;
mitochondrial electron transport chain inhibitor (METI) I acaricides: fenazaquin, pyridaben, tebufenpyrad, tolfenpyrad, flufenerim;
METI II and III substances: acequinocyl, fluacyprim, hydramethylnon;
decouplers: chlorfenapyr;
inhibitors of oxidative phosphorilation: cyhexatin, diafenthiuron, fenbutatin-oxide, propargite;
insect ecdysis inhibitors: cryomazine;
inhibitors of ‘mixed function oxidases’: piperonyl butoxide;
sodium channel blockers: indoxacarb, metaflumizone;
others: benclothiaz, bifenazate, cartap, flonicamid, pyridalyl, pymetrozine, sulfur, thiocyclam, flubendiamide, chlorantraniliprole, cyazypyr (HGW86); cyenopyrafen, flupyrazofos, cyflumetofen, amidoflumet, imicyafos, bistrifluron and pyrifluquinazone.

The pesticide is preferably at least one fungicide, specifically from the class of the strobilurins or carboxanilides. The pesticide is especially preferably pyraclostrobin, boscalid or the mixture of pyraclostrobin and boscalid. In a further preferred embodiment, the pesticide comprises boscalid. In a further preferred embodiment, the pesticide comprises boscalid and pyraclostrobin. In a further preferred embodiment, the pesticide comprises fluxapyroxad.

The amount of pesticide in the dispersion depends mainly on the type of application. As a rule, the weight ratio between pesticide and polyurethane will be in the range of from 1:100 to 1:1 and in particular in the range of from 1:80 to 1:2 and specifically in the range of from 1:50 to 1:5.

Usually, the dispersion has a viscosity (true viscosity measured at 25° C. and a shear rate of 100 s⁻¹) in the range of from 2 to 500 mPas, preferably from 5 to 100 mPas and in particular from 10 to 50 mPas.

In most cases, the dispersion comprises formulation auxiliaries, the choice of auxiliary usually depending on the specific use or the pesticide. Examples of suitable auxiliaries are solvents, surface-active substances (such as surfactants, solubilizers, protective colloids, wetters and adhesives), organic and inorganic thickeners, antifrost agents, antifoams, optionally colorants and stickers (for example for the treatment of seed).

Surface-active substances (adjuvants, wetters, adhesives, dispersants or emulsifiers) which are suitable are the alkali metal, alkaline-earth metal, ammonium salts of aromatic sulfonic acids, for example of lignosulfonic acid (Borresperse® types, Borregaard, Norway), phenolsulfonic acid, naphthalene sulfonic acid (Morwet® types, Akzo Nobel, USA) and dibutylnaphthalenesulfonic acid (Nekal® types, BASF, Germany), and of fatty acids, alkyl- and alkylarylsulfonates, alkyl ether, lauryl ether and fatty alcohol sulfates, and salts of sulfated hexa-, hepta- and octadecanols and of fatty alcohol glycol ethers, condensates of sulfonated naphthalene and its derivatives with formaldehyde, condensates of naphthalene or of the naphthalenesulfonic acids with phenol and formaldehyde, polyoxyethylene octylphenol ether, ethoxylated isooctyl-, octyl- or nonylphenol, alkylphenyl polyglycol ethers, tributylphenyl polyglycol ethers, alkylaryl polyether alcohols, isotridecyl alcohol, fatty alcohol/ethylene oxide condensates, ethoxylated castor oil, polyoxyethylene or polyoxypropylene alkyl ethers, lauryl alcohol polyglycol ether acetate, sorbitol esters, lignin-sulfite liquors and proteins, denatured proteins, polysaccharides (for example methylcellulose), hydrophobe-modified starches, polyvinyl alcohol (Mowiol® types, Clariant, Switzerland), polycarboxylates (Sokalan® types, BASF, Germany), polyalkoxylates, polyvinylamine (Lupamin® types, BASF, Germany), polyethyleneimine (Lupasol® types, BASF, Germany), polyvinylpyrrolidone and their copolymers.

Suitable surfactants are, in particular, anionic, cationic, nonionic and amphoteric surfactants, block polymers and polyelectrolytes. Suitable anionic surfactants are alkali metal, alkaline-earth metal or ammonium salts of sulfonates, sulfates, phosphates or carboxylates. Examples of sulfonates are alkylarylsulfonates, diphenylsulfonates, alpha-olefinsulfonates, sulfonates of fatty acids and oils, sulfonates of ethoxylated alkylphenols, condensed naphthaline sulfonates, sulfonates of dodecyl- and tridecylbenzenes, sulfonates of naphthalenes and alkylnaphthalenes, sulfosuccinates or sulfosuccinamates. Examples of sulfates are sulfates of fatty acids and oils, of ethoxylated alkylphenols, of alcohols, of ethoxylated alcohols, or of fatty acid esters. Examples of phosphates are phosphate esters. Examples of carboxylates are alkyl carboxylates and carboxylated alcohol or alkylphenol ethoxylates.

Suitable nonionic surfactants are alkoxylates, N-alkylated fatty acid amides, aminoxides, ester- or sugar-based surfactants. Examples of alkoxylates are compounds such as alcohols, alkylphenols, amines, amides, arylphenols, fatty acids or fatty acid esters which have been alkoxylated. Ethylene oxide and/or propylene oxide, preferably ethylene oxide, may be applied for the alkoxylation reaction. Examples of N-alkylated fatty acid amides are fatty acid glucamides or fatty acid alkanolamides. Examples of esters are fatty acid esters, glycerol esters, or monoglycerides. Examples of sugar-based surfactants are sorbitans, ethoxylated sorbitans, sucrose and glucose esters or alkylpolyglucosides. Suitable cationic surfactants are quaternary surfactants, for example quaternary ammonium compounds with one or two hydrophobic groups, or salts of long-chain primary amines. Examples of amphoteric surfactants are alkylbetaines and imidazolines. Suitable block polymers are block polymers of the A-B or of the A-B-A type comprising blocks of polyethylene oxide and polypropylene oxide or of the A-B-C type comprising alkanol, polyethylene oxide and polypropylene oxide. Suitable polyelectrolytes are polyacids or polybases. Examples of polyacids are alkali metal salts of polyacrylic acid. Examples of polybases are polyvinylamines or polyethyleneamines.

Preferably, the dispersion comprises less than 10% by weight, especially preferably less than 7% by weight, in particular less than 5% by weight and specifically less than 2% by weight total amount of nonionic surfactants. To calculate this nonionic surfactant content, nonionic surfactants which have been added for other purposes, such as adjuvants (such as alcohol alkoxylates) or spreaders (such as alkoxylated alcohols) are also included in the calculation.

Examples of adjuvants are organic-modified polysiloxanes, such as BreakThruS 240®; alcohol alkoxylates, such as Atplus®245, Atplus®MBA 1303, Plurafac®LF and Lutensol® ON; EO-PO block polymers, for example Pluronic® RPE 2035 and Genapol® B; alcohol ethoxylates, for example Lutensol® XP 80; and sodium dioctyl sulfosuccinate, for example Leophen® RA.

Examples of thickeners (i.e. compounds which impart a modified flow behavior to the composition, i.e. high viscosity at rest and low viscosity in the agitated state) are polysaccharides such as xanthan (Kelzan®, CP Kelco Inc; Rhodopol® 23, Rhodia), inorganic layered minerals, such as magnesium aluminum silicates (Veegum® types, R. T. Vanderbilt; attapulgite from Attaclay), or organo-layered silicates, such as smectites after treated with quaternary ammonium salts.

Film-forming adjuvants may be added to improve film formation, in particular at low temperatures, upon application. Examples of film-forming adjuvants are volatile hydrocarbons such as petroleum fractions, white mineral oils, liquid paraffins, glycols such as butylene glycol, ethylene glycol, diethylene glycol and propylene glycol, glycol ethers such as glycol butyl ether, diethylene glycol monobutyl ether (butyl diglycol), 1-methoxy-2-propanol, dipropylene glycol methyl ether, dipropylene glycol propyl ether, dipropylene glycol-n-butyl ether, tripropylene glycol-n-butyl ether, 2,3-phenoxypropanol, glycol esters and glycol ether esters such as butyl glycol acetate, diethylene glycol mono-n-butyl ether acetate, 2,2,4-trimethylpentane-1,3-diol monoisobutyrate, butyl glycol diacetate, methoxypropyl acetate. Preferred film-forming adjuvants are glycol ethers, glycol esters and glycol ether esters, in particular butyl diglycol and methoxypropyl acetate.

Examples of suitable antifreeze agents are ethylene glycol, 1,2-propylene glycol, urea and glycerol, preferably glycerol and 1,2-propylene glycol. Examples of antifoams are silicone emulsions (such as, for example, Silikon® SRE, Wacker, Germany or Rhodorsil®, Rhodia, France), long-chain alcohols, fatty acids, salts of fatty acids, organofluorine compounds and their mixtures. Examples of stickers are polyvinylpyrrolidone, polyvinyl acetate, polyvinyl alcohol and cellulose ethers (Tylose®, Shin-Etsu, Japan).

The aqueous, pesticide-comprising dispersion of a polyurethane can be applied to any plants or plant parts. This means that the plant which grows on the cropping area can be treated (for example by extensive spraying of a cropping area or by the targeted application to a zone on a plant, such as the grapevine), or that plant parts which have been separated from the plant may be treated. Examples of plant parts which have been separated from the plant are seeds, roots, fruits, tubers, bulbs, parts of stems, parts of branches, and rhizomes.

It is possible to treat any type of plants or plant parts which are obtained from any type of plants. Examples are cereals, beet, fruit, legumes, soybeans, oilseed rape, mustard, olives, sunflowers, coconut, cucurbits, cotton, citrus fruit, vegetable plants, maize, sugar cane, oil palm, tobacco, coffee, tea, bananas, grapevines, hops, grass, rubber plants, ornamentals, forestry plants. Plants which may be used include those which, as the result of breeding, including genetic engineering methods, are tolerant to attack by insects, viruses, bacteria or fungi or to the application of herbicide. Preferred types of plants are woody plants, in particular fruit trees, such as plum, peach, cherry, apple, pear, mirabelles and specifically grapevines. It is possible to treat grapevines of any grape varieties, such as white grapevine varieties and red grapevine varieties, for example Müller-Thurgau, Bacchus, Riesling, Scheurebe, Silvaner or Dornfelder, Lemberger, Tempranillo and Trollinger as red grapevine varieties. In a further preferred embodiment plants are oil palms.

Depending on the fungicidal active substance which is present in the composition, the composition can be used for protecting the woody plant from infection by the following fungal pathogens or for the treatment of an infection with these fungal pathogens and/or a disease caused thereby: Botryosphaeria species, Cylindrocarpon species, Eutypa lata, Neonectria liriodendri and Stereum hirsutum, Ascomycetes, Deuteromycetes, Basidiomycetes, Peronosporomycetes (syn. Oomycetes), and Fungi imperfecti, Ascomycetes such as Ophiostoma spp., Ceratocystis spp., Aureobasidium pullulans, Sclerophoma spp., Chaetomium spp., Humicola spp., Petriella spp., Trichurus spp; Basidiomycetes such as Coniophora spp., Coriolus spp., Gloeophyllum spp., Lentinus spp., Pleurotus spp., Poria spp., Serpula spp. and Tyromyces spp., Deuteromycetes such as Aspergillus spp., Cladosporium spp., Penicillium spp., Trichoderma spp., Alternaria spp., Paecilomyces spp. and Zygomycetes such as Mucor spp., Glomerella cingulata, Guignardia budelli, Isariopsis clavispora, Phomopsis species, for example P. viticola, Plasmopara viticola, Pseudopezicula tracheiphilai, Erysiphe (syn. Uncinula) necator, Ascomycetes, Deuteromycetes, Basidiomycetes, Peronosporomycetes (syn. Oomycetes) and Fungi imperfecti.

In one embodiment, the present invention is particularly suitable for the protection against, and for the treatment of diseases caused by: Phaeomoniella chlamydospora, aleophilum, parasiticum, Phaeoacremonium spp. (aleophilum, inflatipes, chlamydosporum, angustius, viticola, rubrigenum, parasiticum), Formitipora mediterranea (syn. Phellinus punctatus, Phellinius igniarius Fomitiporia punctata), Eutypa lata, Eutypa armeniacae, Libertella blepharis, Stereum hirsutum, Phomopsis viticola, amygdalii, Botryosphaeria spp. (australis, dothidea, obtusa, stevensii, parva, rhodina), Cylindrocarpon spp. (destructans, optusisporum), Campylocarpon spp., Guignardia bidwellii, rubrigenum), Elsinoe ampelina, Verticilium, Armillaria mellea, Clitopilus hobsonii, Flammulina velutipes, Pleurotus pulmonarius, Inonotus hispidus, Trametes hirsuta, Trametes versicolor, Peniphora incarnate, Hirneola auriculae judge, Diaporthe helianthi, ambigua, Pleurostomophora sp., Cadophora sp., Phialemonium sp.

In one embodiment, the dispersion according to the invention is particularly suitable for the protection against, and the control of, Elsinoe ampelina in grapevines. In a preferred embodiment, the dispersion is used for the protection of woody plants, specifically grapevines, against Esca, i.e. for the protection of woody plants, specifically grapevines, against infection with the complex of pathogens which are associated with Esca disease. The dispersion can also be used for the treatment of Esca in woody plants, specifically grapevines, or for the treatment of woody plants which are infected with the pathogens which cause Esca. As already explained above, in central Europe, this disease is frequently caused by the main pathogens Phaeomoniella chlamydospora, Phaeoacremonium spp. (aleophilum, inflatipes, chlamydosporum), and Formitipora mediterranea (syn. Phellinus punctatus, Fomitiporia punctata). In this case, the dispersion preferably comprises at least one strobilurin, in particular pyraclostrobin, optionally in combination with at least one further fungicide, in particular boscalid.

The invention furthermore relates to the use of a pesticide for the treatment of Esca in woody plants (specifically grapevines), where the pesticide comprises pyraclostrobin and boscalid. The weight ratios of pyraclostrobin to boscalid can vary within wide ranges, for example from 100 to 1 up to 1 to 100. It is preferably in the range of from 10 to 1 up to 1 to 15, especially preferably 3 to 1 up to 1 to 6, and in particular 1 to 1 up to 1 to 3. In a further preferred embodiment, pyraclostrobin and boscalid are present in a synergistically active weight ratio. The pesticide can be used at any ready-to-use concentrations, for example at a concentration of from 0.01 to 100 g/l pyraclostrobin and from 0.02 to 200 g/l boscalid, preferably 0.1 to 10 g/l pyraclostrobin and 0.2 to 20 g/l boscalid, especially preferably at a concentration of 0.3 to 3 g/l pyraclostrobin and 0.5 to 5 g/l boscalid. The application rate of these ready-to-use concentrations can amount to 1 to 300 l/ha, preferably 20 to 150 l/ha, especially preferably 30 to 90 l/ha.

The application of an aqueous, pesticide-comprising dispersion of a polyurethane to plants or plant parts can be effected in a customary manner and depends in the known manner on the type of the plants or plant parts to be treated or to be protected. The application can be effected by dabbing, painting, dipping, brushing on or spraying, preferably by spraying. Usually, the polyurethane is applied to the surface, whereby the pesticide and optionally the polyurethane penetrate the surface zone. The polyurethane, in turn, forms a permanently elastic continuous layer or a film on or in the surface and in this manner prevents plant pathogens from penetrating. The resulting polyurethane layer is weatherproof, frost-resistant, UV-resistant, rainfast, abrasion proof and nontoxic to the plant. Upon application, good penetration depths of the pesticide into the plant material are achieved, penetration preferably taking place in the direction of the vascular bundles. Frequently, the depth of penetration is at least 0.2 cm, in particular at least 0.5 cm and particularly preferably at least 1 cm, down to 2.5 cm or 3 cm or deeper. Application is preferably effected at temperatures in the range of from −10° C. to +50° C., particularly preferably in the range of from −5° C. to +20° C. and very particularly preferably in the range of from −3° C. to +10° C.

The wounds to be treated or to be protected may take the form of natural injuries as they arise as the result of windbreak, frost or other atmospheric influences, or they may in particular take the form of the wound areas caused by pruning. They may be wounds in the bark zone, or else wounds in the cross-section of the wood, i.e. wounds caused by sawing or cutting.

In accordance with a preferred embodiment, the application is effected by spraying the dispersion. The term “spraying” also comprises the nebulizing, blowing and splashing-on of the composition. The equipment used for spraying may be customary equipment such as, for example, commercially available atomizers, spraying apparatus, manual sprayers, and pneumatic or manual pruning shears with spray function by means of which the dispersion can be applied in a targeted manner to pruning wounds within the scope of the usual spraying procedure. The application can be effected in a targeted manner in the wound zone, or the dispersion can be applied over a large area of the plant or parts of the plant. In accordance with an especially preferred embodiment of the invention, the application is effected by what is known as tunnel spraying, where, in plantations of fruit trees or grapevines, the woody parts after a pruning treatment are sprayed in a targeted manner in the pruning zone with a dispersion, optionally after dilution, and excess spray liquor is collected. In this manner, the pruning sites and surrounding woody parts are treated.

In one embodiment, the dispersion according to the invention is used in a multi-step method. Thus, for example, it is possible to apply, to the surface to be treated or to be protected, in a first pass, a first plant protectant, in particular a fungicide, or an active ingredient preparation of this active ingredient, and the dispersion is then applied in one of the subsequent passes in the manner described herein.

The invention furthermore relates to an aqueous pesticide-comprising dispersion of a polyurethane, which is a reaction product of at least one polyol (A) and at least one polyisocyanate (B), wherein the polyisocyanate comprises at least 10% by weight of aromatic diisocyanate and at least 10% by weight of aliphatic diisocyanate, in each case based on the polyisocyanate. The polyol (A) preferably comprises a polyester polyol which is composed of aliphatic diols and aliphatic dicarboxylic acids. The polyester polyol preferably has a molecular weight of from 500 to 6000. The polyurethane is preferably a reaction product of (A), (B) and at least one salt (C) of an aminocarboxylic acid or an aminosulfonic acid. The salt (C) preferably comprises an adduct of ethylenediamine and unsaturated, aliphatic carboxylic acid salts. Further preferred embodiments of the aqueous pesticide-comprising dispersion of the polyurethane are as described above.

Before application, the aqueous pesticide-comprising dispersion of a polyurethane can be diluted, for example with water in order to obtain what is known as a tank mix. However, the dispersion may also be applied as such. Usually, the tank mix is prepared by diluting the dispersion to the 2- to 100-fold, preferably the 5- to 40-fold, and in particular the 10- to 20-fold volume. Oils of various types, and wetters, adjuvants, further pesticides may be added to the tank mix or else only immediately prior to preparing the tank mix from the dispersion. These agents can be admixed in the weight ratio agent to dispersion 1:100 to 100:1, preferably 1:10 to 10:1.

The dispersion usually comprises water in a concentration of from 250 to 850 g/l, preferably 350 to 750 g/l and in particular 450 to 650 g/l.

The dispersion usually comprises polyurethane in a concentration of from 100 to 650 g/l, preferably 200 to 550 g/l and in particular 300 to 450 g/l. These concentration data do not refer to the aqueous dispersion of the polyurethane, but to the polyurethane itself.

The dispersion usually comprises pesticide in a concentration of from 0.01 to 300 g/l, preferably 0.5 to 100 g/l, in particular 2 to 50 g/l.

The dispersion usually comprises surface-active substances in a concentration of from 0.001 to 40 g/l, preferably 0.01 to 25 g/l, in particular 0.05 to 5 g/l.

The dispersion usually comprises thickeners in a concentration of from 0.001 to 5 g/l, preferably 0.01 to 0.5 g/l.

The dispersion usually comprises antifreeze agent in a concentration of from 0.05 to 350 g/l, preferably 0.1 to 250 g/l, in particular 0.5 to 150 g/l.

The dispersion may optionally comprise film-forming adjuvants in a concentration of from 10 to 250 g/l, preferably 50 to 150 g/l.

The dispersion may optionally comprise spreading agents in a concentration of from 0.1 to 250 g/l, preferably 1 to 150 g/l, and in particular 5 to 50 g/l. Suitable spreading agents are alkoxylated alcohols, the alcohol preferably being an unbranched or branched aliphatic C₆- to C₃₂-monoalcohol and the alkoxylation having been carried out with C₂- to C₆-alkylene oxide, preferably C₂-alkylene oxide.

The invention furthermore relates to the use of the dispersion according to the invention for controlling phytopathogenic fungi and/or undesired plant growth and/or undesired insect or mite attack and/or for regulating the growth of plants, by allowing the dispersion to act on the respective pests, their environment and/or the plants or plant parts to be protected from the respective pests, the soil and/or on undesired plants and/or the use plants and/or their environment. Allowing to act is usually done by applying the dispersion.

The invention furthermore relates to plant parts which have been separated from a plant and to which the dispersion according to the invention has been applied. Suitable plant parts, plants and dispersions are as described above. The application can be effected as described above. Preferred are plant parts which have been separated from a plant and to which the dispersion according to the invention has been applied, where the plant part comprises the dispersion.

Advantages of the method according to the invention are that the dispersion can be applied in a simple manner, for example by spraying. The dispersion even forms a film at low temperatures, for example below 20° C. The dispersion is storage-stable over a prolonged period, including at elevated temperatures, and can be produced inexpensively on a large scale. A further advantage is that the dispersion is stable even at low concentrations of surface-active substances, which reduces environmental pollution as a result of the surface-active substances. The protective film which is formed after the dispersion of the polyurethane has been applied has good and durable adherence. The method is furthermore highly suitable for the protective treatment of fungal diseases on woody plants, specifically for the treatment of Esca in grapevines. It is furthermore advantageous for the curative treatment of fungal diseases on woody plants.

The following examples and figures are intended to illustrate the invention.

### EXAMPLE 1

### Preparation of a Polyurethane Dispersion

### Polyurethane Dispersion A

150 parts by weight of a polyester of adipic acid, hexanediol and neopentyl glycol with an OH number of 55 was dehydrated in a stirred flask at 130° C./20 torr in the course of 30 min The polyester was cooled, dissolved in 145 parts of acetone and treated with 30 parts of butane-1,4-diol. Then, a mixture of 50 parts of toluene diisocyanate (isomer ratio 2.4/2.6=80/20) and 25 parts of hexamethylene diisocyanate and also 0.015 part of dibutyltin dilaurate was added. After stirring for 3 hours at 60° C., the mixture was diluted with 220 parts of acetone and cooled to room temperature. 14 parts of a 40% strength aqueous solution of an equimolar adduct of ethylenediamine and sodium acrylate was stirred into the resulting solution of the prepolymer. After 20 minutes, 370 parts of water were added dropwise, and the acetone was then distilled off under reduced pressure. This gave a finely divided, stable dispersion which did not sediment even upon storage for one year at room temperature. The solids content was adjusted to 40% by weight with water.

### Polyurethane Dispersion B

120 parts by weight of a polyester of adipic acid and ethylene glycol with a molecular weight of 2000 g/mol was dehydrated in a stirred flask. The polyester was cooled, dissolved in 65 parts of acetone and treated with 35 parts of neopentyl glycol. Then, 95 parts of 4,4′-diisocyanatodiphenylmethane and 13 parts of isophorone diisocyanate are added, with stirring, and stirring was continued for one hour. The composition is diluted with 260 parts of acetone, cooled to room temperature, and 40 parts of a 40% strength aqueous solution of the equimolar adduct of ethylenediamine and sodium acrylate are stirred in. After 30 minutes, 400 parts of demineralized water were slowly added dropwise, and the acetone was stripped off under reduced pressure. This gave a finely divided, stable dispersion (solids content approximately 40% by weight) which did not sediment even upon storage for three months.

### Polyurethane Dispersion C

150 parts of a polyester of adipic acid, hexanediol and neopentyl glycol with a mean molecular weight of 2000 g/mol were dehydrated in a stiffed flask and treated with 34 parts of butane-1,4-diol, 70 parts of acetone and 70 parts of toluene diisocyanate, and the mixture was stirred for 90 min when the acetone was boiling. Then, 13 parts of hexamethylene diisocyanate and 0.1 part of dibutyltin dilaurate were added, and stirring was continued for 90 min Thereafter, the mixture was diluted with 270 parts of acetone, and 19 parts of sodium lysinate were stirred in at 40° C. After 20 min, 370 parts of water were slowly added, with stirring, and the acetone was distilled off under reduced pressure. This gave a finely-divided, highly-stable dispersion with a solids content of 40% by weight. After the dispersion had been left to stand for 6 months at 20° C., no sediment had formed.

### EXAMPLE 2

### Preparation of Suspension Concentrates of the Pesticide

### a) Suspension Concentrate SC1

An aqueous suspension concentrate comprising 200 g/l of boscalid and 100 g/l of pyraclostrobin was prepared. It comprised 35 g/l of a polyethylene-glycol-comprising dispersant, 15 g/l of sulfate-comprising dispersant, 100 g/l of glycerol, 2 g/l of xanthan as thickener, 2 g/l of bactericide and 5 g/l of silicone-comprising antifoam.

### b) Suspension Concentrate SC2

An aqueous suspension concentrate comprising 400 g/l of pyraclostrobin was prepared. It comprised 30 g/l of a polyethylene glycol-comprising dispersant, 20 g/l of nonionic propylene glycol-based surfactant, 70 g/l of an antifreeze agent, 2 g/l of xanthan as thickener, 2 g/l of bactericide and 5 g/l of silicone-comprising antifoam.

### c) Suspension Concentrate SC3

An aqueous suspension concentrate comprising 500 g/l of boscalid was prepared. It comprised 20 g/l of a polyethylene glycol-comprising dispersant, 30 g/l of a nonionic propylene-glycol-based surfactant, 70 g/l of an antifreeze agent, 2 g/l of thickener, 2 g/l of bactericide and 5 g/l of silicone-comprising antifoam.

### EXAMPLE 3

### Preparation of a Pesticide-Comprising Dispersion of a Polyurethane

The pesticide-comprising suspension concentrate SC1 was mixed with the polyurethane dispersion of Example lA (specific gravity 1.06 kg/l) in the specified amounts (Table 1). This gave a stable concentrate of the dispersion. The samples were stored for four weeks at 50° C., and the dispersion was still stable. A nonionic alkyl polyethylene glycol ether was used as surfactant A.

TABLE 1

| No. | Concentrate [l] | Polyurethane [l] | Adjuvant [l] |
|---|---|---|---|
| 1 | 0.5 l of SC1 | 9.5 | — |
| 2 | 0.5 l of SC1 | 4.5 | — |
| 3 | 0.5 l of SC1 | 8.5 | 1 1 1,2-propylene glycol |
| 4 | 0.5 l of SC1 | 4.0 | 0.5 1 2-(2-butoxyethoxy)ethanol |
| 5 | 0.5 l of SC1 | 3.9 | 0.5 1 1,2-propylene glycol |
| | | | 0.1 1 surfactant A |

### EXAMPLE 4

### Preparation of a Dilute Dispersion (Tank Mix)

The pesticide-comprising suspension concentrate SC1, SC2 or SC3 was mixed with the polyurethane dispersion of Example 1A in the specified amounts and diluted to a total volume of 50 l with water (Table 2). The resulting dilute dispersions were capable of being sprayed onto grapevines using commercially available spraying apparatuses. The typical application rate was 50 l per hectare.

TABLE 2

| No. | Concentrate [l] | Polyurethane [l] | Adjuvant [l] |
|---|---|---|---|
| 1^{a)} | — | 9.5 | — |
| 2 | 0.5 l of SC1 | 9.5 | — |
| 3 | 0.375 l of SC1 | 7.125 | — |
| 4 | 0.25 l of SC1 | 4.75 | — |
| 5 | 0.125 l of SC2 | 9.875 | — |
| 6 | 0.2 l of SC3 | 9.8 | — |
| 8 | 0.5 l of SC1 | 8.5 | 1 1 1,2-propylene glycol |
| 9 | 0.5 l of SC1 | 4.0 | 0.5 1 2-(2-butoxyethoxy)ethanol |
| 10 | 0.5 l of SC1 | 3.9 | 0.5 1 1,2-propylene glycol |
| | | | 0.1 1 surfactant A |

^{a)}not according to the invention

### EXAMPLE 5

### Field Experiment in Spain

The experiment was carried out in a vineyard in Spain using the variety Chardonnay. For each combination, 20 one-year-old shoots were pruned at the beginning of March above the 6th to 7th burgeon. On the same day, the test product in question was applied to the pruning wounds, using a brush (see Table 3). The pruning wound was inoculated with the pathogen on the next day. The pathogen Botryosphaeria obtuse had previously been propagated in Petri dishes containing potato-glucose agar (PDA) at 25° C. over a period of 7 or 25 days. To carry out the inoculation, mycelium/agar fragments 5 mm in size were excised from the Petri dishes and placed on the pruning wounds of the shoots.

Immediately after the inoculation, the pruning wound was wrapped in Parafilm® M (stretchable film, consists essentially of polyolefins and paraffin waxes) and left there until the experimental series has ended. The vineyard was tended for 5 months according to customary viticultural practice. The shoots of the grapevines were harvested after 5 months and examined in the laboratory. The shoots were scored for necroses. Shoot fragments around the necrotic zone with a thickness of 4 mm were processed for reisolation. The surface was sterilized with alcohol for 4 minutes and subsequently incubated in Petri dishes containing PDA at 25° C. After 3-4 weeks, the frequency of fragments with pathogen infection was determined (Table 3).

The polyurethane dispersion was tested in combination with active substances pyraclostrobin and boscalid at various application rates. The commercially available product Bilko® (SL formulations comprising 40% by weight of quinosol, Probelte S.A.) in a 1% strength solution was used to compare the activity. This product is registered in Spain for protecting pruning wounds against wood diseases.

TABLE 3
Frequency of shoots with Botryosphaeria obtusa infection

| | | | Active | Frequency of |
|---|---|---|---|---|
| | | | substance | pathogen infection |
| No. | Treated with | Inoculated | dose [ppm] | [%] |
| 1^{a)} | — | No | 0 | 0 |
| 2 ^{a)} | — | Yes | 0 | 100 |
| 3 | Ex. 4-2 ^{b)} | Yes | 1000 + 2000 ^{c)} | 35.7 |
| 4 | Ex. 4-3 ^{b)} | Yes | 750 + 1500 ^{c)} | 60.0 |
| 5 | Ex. 4-4 ^{b)} | Yes | 500 + 1000 ^{c)} | 78.6 |
| 6^{a)} | Bilko ® | Yes | 4000 | 100 |

^{a)}Not in accordance with the invention.
^{b)} Example 4, table entries No. 2, 3 or 4.
^{c)} Dose for pyraclostrobin + boscalid.

### EXAMPLE 6

### Field Experiment in Portugal

Two experiments with randomly arranged combinations were carried out in vineyards in Portugal, using the grape varieties Syrah and Touriga Nacional. For each combination, 18 one-year-old shoots were pruned in mid-February, approximately 3-4 cm above the third burgeon. The test product in question was applied to the pruning wounds on the same day, using a brush. The pruning wound was inoculated with pathogen one day later. The pathogen Phaeomoniella chlamydospora had previously been propagated in Petri dishes containing malt agar at 20° C. in the dark. Thereafter, small agar pieces containing conidia of the pathogen were subsequently transferred into Erlenmeyer flasks, and these were placed into a shaker for 14 days at 20° C. in the dark. Thereafter, the liquid culture was filtered, and a concentration of 105 conidia/ml was adjusted using SDW. A 50-μl drop of the conidial solution was applied to each pruning cut, using a pipette.

Immediately after the inoculation, the pruning wound was wrapped with Parafilm® M, which remained there until the end of March. The vineyard was tended until October following customary viticultural practice. The grapevine shoots were harvested and examined in the laboratory at 5 levels: 1) 0.5-1 cm below the pruning wound; 2) 1 cm above the first burgeon; 3) 1 cm below the third burgeon; 4) 1 cm above the second burgeon; 5) 1 cm below the second burgeon. In each case 5 small tissue pieces were removed from the shoots at the 5 levels and placed on PDA supplemented with chloamphenical and incubated for several weeks at 20° C. The frequency of the fragments with pathogen infection was determined (Tables 4 and 5).

The polyurethane dispersion was tested not only on its own (formulation 6 A: polyurethane dispersion A of Example 1, diluted with water to 210 g/l polymer), but also in combination with the active substances pyraclostrobin (formulation 6 B: polyurethane dispersion A of Example 1 and suspension concentrate SC2 of Example 2, diluted with water to 210 g/l polymer and 1.0 g pyraclostrobin) or boscalid (formulation 6 C: polyurethane dispersion A of Example 1 and suspension concentrate SC3 of Example 2, diluted with water to 210 g/l polymer and 2.0 g boscalid). The standard used was Escudo® (suspoemulsion of 5 g/l flusilazole and 10 g/l carbendazim, commercially available from Dupont), which is registered in some countries against wood diseases in grapevines.

TABLE 4
Frequency of the shoots with Phaeomoniella chlamydospora infection
(conidia) in the variety Syrah

| | | | | Frequency of |
|---|---|---|---|---|
| | | | Active substance | pathogen infection |
| No. | Treated with | Inoculated | dose [ppm] | [%] |
| 1^{a)} | — | No | 0 | 0 |
| 2^{a)} | — | Yes | 0 | 100 |
| 3 | 6 B | Yes | 1000 | 55.6 |
| 4 | 6 C | Yes | 2000 | 62.5 |
| 5 | 6 A | Yes | 0 | 44.4 |
| 6^{a)} | Escudo ® | Yes | 15000 | 66.7 |

^{a)}not in accordance with the invention.

TABLE 5
Frequency of the shoots with Phaeomoniella chlamydospora infection
(conidia) in the variety Touriga Nacional

| | | | | Frequency of |
|---|---|---|---|---|
| | | | Active substance | pathogen infection |
| No. | Treated with | Inoculated | dose [ppm] | [%] |
| 1^{a)} | — | No | 0 | 0 |
| 2^{a)} | — | Yes | 0 | 100 |
| 3 | 6 B | Yes | 1000 | 61.1 |
| 4 | 6 C | Yes | 2000 | 50.0 |
| 5 | 6 A | Yes | 0 | 64.7 |
| 6^{a)} | Escudo ® | Yes | 15000 | 52.9 |

^{a)}not in accordance with the invention.

### EXAMPLE 7

### Field Experiment in Germany

The experiments were carried out in a vineyard using the variety Riesling. For each combination, 20 one-year-old shoots were pruned in mid-March 2-3 cm above the third burgeon. The product in question was applied to the pruning wounds on the same day, using a brush. 7 days later, the pruning wounds were inoculated with a spore solution of Phaeomoniella chlamydospora or Phaeoacremonium aleophilium (40 μl at a concentration of 105 conidia/ml). The grapevine shoots were harvested mid-October, cut transversely at various positions of the shoot and scored for browning inside the shoot in four classes (class 1=no symptoms; 2=individual dots, no gummosis; 3=dark ring segments, some gummosis; 4=dark ring symptoms, pronounced gummosis). The disease level (Table 6) was calculated using the following formula: disease=(cl1+cl2*2+cl3*3+cl4*4)/(cl1+cl2+cl3+cl4), where cl is the number of samples in the respective classes 1-4.

TABLE 6
Disease level of the frequency of shoots with infection of Phaeomoniella
chlamydospora or Phaeoacremonium aleophilum

| | | | Active substance | Disease | Disease |
|---|---|---|---|---|---|
| No. | Treated with | Inoculated | dose [ppm] | level PC ^{e)} | level PA ^{f)} |
| 1^{a)} | — | No | 0 | 1.15 | 1.15 |
| 2^{a)} | — | Yes | 0 | 2.39 | 2.68 |
| 3 | Ex. 4-2 ^{b)} | Yes | 1000 + 2000 ^{c)} | 1.39 | 1.92 |
| 4 | Ex. 4-3 ^{b)} | Yes | 750 + 1500 ^{c)} | 1.26 | 1.75 |
| 5 | 6 C ^{d)} | Yes | 2000 | 1.28 | 1.44 |
| 6 | 6 A ^{d)} | Yes | 0 | 1.26 | 1.53 |

^{a)}not in accordance with the invention.
^{b)} Example 4, able entries No. 2 or 3.
^{c)} Dose for pyraclostrobin + boscalid.
^{d)} Formulations see Example 6.
^{e)} Phaeomoniella chlamydospora.
^{f)} Phaeoacremonium aleophilum.

### EXAMPLE 8

### Greenhouse Experiment

The experiments were carried out in the greenhouse using scions of the variety Willer Thurgau in pots. One experiment was designed as a curative experiment and the second one as a preventative experiment. For each combination, 10 plants in pots were allowed to take and then pruned in mid-June 2 cm above the second burgeon. Both experiments were inoculated after two days using a conidia solution of the pathogen Phaeomoniella chlamydospora (40 μl of a concentration of 105 conidia/ml). In the preventative experiment, the polyurethane products were applied after two days and in the curative experiment after 5 days. The plants were harvested in mid-October or at the end of January, cut transversally at various positions of the shoot and scored for browning in the shoot in 4 classes as described in Example 6 (for results see Table 7).

TABLE 7
Disease level of the frequency of shoots with infection of Phaeomoniella
chlamydospora (preventative or curative treatment)

| | | | Active | Disease | Disease |
|---|---|---|---|---|---|
| | Treated | Inocu- | substance | level | level |
| No. | with | lated | dose [ppm] | PRE ^{e)} | CUR ^{e)} |
| 1^{a)} | — | No | 0 | 1.20 | 1.20 |
| 2^{a)} | — | Yes | 0 | 2.55 | 2.55 |
| 3 | Ex. 4-2 ^{b)} | Yes | 1000 + 2000 ^{c)} | 1.20 | 1.85 |
| 4 | 6 B ^{d)} | Yes | 1000 | 2.28 | 2.40 |
| 5 | 6 C ^{d)} | Yes | 2000 | 1.65 | 1.95 |
| 6 | 6 A ^{d)} | Yes | 0 | 1.90 | 2.00 |

^{a)}not in accordance with the invention.
^{b)} Example 4, table entries No. 2 or 3.
^{c)} Dose for pyraclostrobin + boscalid.
^{d)} Formulations see Example 6.
^{e)} PRE = preventative treatment, CUR = curative treatment.

1-14. (canceled)

15. An aqueous, pesticide-comprising dispersion of a polyurethane which is a reaction product of at least one polyol (A) and at least one polyisocyanate (B), wherein the polyisocyanate comprises at least 10% by weight of aromatic diisocyanate and at least 10% by weight of aliphatic diisocyanate, in each case based on the polyisocyanate.

16. The dispersion of claim 15, wherein the polyol (A) comprises a polyester polyol which is composed of aliphatic diols and aliphatic dicarboxylic acids.

17. The dispersion of claim 16, wherein the polyester polyol has a molecular weight of from 500 to 6000.

18. The dispersion of claim 15, wherein the polyurethane is a reaction product of (A), (B) and at least one salt (C) of an aminocarboxylic acid or an aminosulfonic acid.

19. The dispersion of claim 18, wherein the salt (C) comprises an adduct of ethylenediamine and unsaturated, aliphatic carboxylic acid salts.

20. A method of protecting plants comprising applying an aqueous, pesticide-comprising dispersion of a polyurethane which is a reaction product of at least one polyol (A) and at least one polyisocyanate (B), to plants or plant parts, wherein the polyisocyanate comprises at least 10% by weight of aromatic diisocyanate and at least 10% by weight of aliphatic diisocyanate, in each case based on the polyisocyanate.

21. The method of claim 20, wherein the polyol (A) comprises a polyester polyol which is composed of aliphatic diols and aliphatic dicarboxylic acids.

22. The method of claim 21, wherein the polyester polyol has a molecular weight of from 500 to 6000.

23. The method of claim 20, wherein the polyurethane is a reaction product of (A), (B) and at least one salt (C) of an aminocarboxylic acid or an aminosulfonic acid.

24. The method of claim 23, wherein the salt (C) comprises an adduct of ethylenediamine and unsaturated, aliphatic carboxylic acid salts.

25. The method of claim 20, wherein the plants are grapevines.

26. A method for controlling phytopathogenic fungi, undesired plant growth and/or undesired insect or mite attack and/or for regulating the growth of plants comprising allowing the dispersion of claim 15 to act on the respective pests, their environment and/or the plants or plant parts to be protected from the respective pests, the soil and/or on undesired plants and/or the use plants and/or their environment.

27. The method of claim 26, wherein the polyol (A) comprises a polyester polyol which is composed of aliphatic diols and aliphatic dicarboxylic acids.

28. The method of claim 27, wherein the polyester polyol has a molecular weight of from 500 to 6000.

29. The method of claim 26, wherein the polyurethane is a reaction product of (A), (B) and at least one salt (C) of an aminocarboxylic acid or an aminosulfonic acid.

30. The method of claim 29, wherein the salt (C) comprises an adduct of ethylenediamine and unsaturated, aliphatic carboxylic acid salts.

31. The method of claim 26, wherein the plants are grapevines.

32. A method for treating Esca in woody plants comprising applying to woody plants a pesticide comprising pyraclostrobin and boscalid.

33. The method of claim 32, wherein the weight ratio of pyraclostrobin to boscalid is in the range of from 10 to 1 up to 1 to 15.

34. The method of claim 32, wherein the pesticide is used at a ready-to-use concentration of from 0.1 to 10 g/l pyraclostrobin and from 0.2 to 20 g/l of boscalid.

35. A plant part which has been separated from a plant and to which the dispersion according to claim 15 has been applied.
